(19) United States
(12) Patent Application Publication
(10) Pub. No.: US 2021/0213084 A1
(43) Pub. Date: Jul. 15, 2021

(54) A COMPOSITION CONTAINING CASHEW LEAF EXTRACT AND PRODUCTION METHOD THEREOF

(71) Applicant: MARK ONE INNOVATION CENTER COMPANY LIMITED, Samutprakarn (TH)

(72) Inventor: Petoon MANEEPAIROJ, Bangkok (TH)

(21) Appl. No.: 17/258,262

(22) PCT Filed: Aug. 1, 2019
(86) PCT No.: PCT/TH2019/000025

§ 371 Date: Jan. 6, 2021

(51) Int. Cl. A61K 36/22; A61K 47/12; A61K 47/36; A61K 9/48; A61K 9/00; A61K 9/68; A23L 33/105; A23L 33/125; A23L 33/00; A23L 2/52

(52) U.S. Cl. CPC A61K 36/22; A61K 47/12; A61K 47/36; A61K 9/4833; A61K 9/0056; A61K 9/0058; A23V 2002/00; A23L 33/105; A23L 33/125; A23L 33/40; A23L 2/52; A61K 2236/331; A61K 2236/333; A61K 9/0095

(57) ABSTRACT

A composition containing cashew leaf extract comprises at least cashew leaf extract, magnesium stearate and maltodextrin wherein the said cashew leaf extract is extracted by water or alcohol or the combination of alcohol and water or other organic solvents or combination thereof, preferably the hydroalcoholic extracts. The composition containing cashew leaf extract can be used as the tonic and/or adaptogenic agents, for enhancing the efficiency of male sexual functions, for enhancing male sexual fertility, for improving cognitive functions. The composition containing cashew leaf extract according to this invention is aimed to increase the commercialization value of cashew leaf which is rich of the health benefit from several nutrient contained in the cashew leaf such as increasing the function of neurological system related to monoamines especially dopamine which increase male sexual functions and also improving the cognitive function.

### FIELD OF THE INVENTION

The present disclosed invention relates to the field of the composition containing cashew leaf (Anacardium occidentale) extract which the said composition can be used for dietary supplements for increasing testosterone levels in humans and improving sexual performance and anti-stress.

### BACKGROUND OF INVENTION

Cashew (Anacardium occidentale Linn) is a tropical plant found in Latin America and South East Asia. Cashew leaf is commonly used as vegetable in Thailand due to the special taste and high vitamin C and beta-carotene content. In Western Africa and Latin America, the cashew leaf has been used as a traditional medicine for treating sexual dysfunction and sexual transmitted diseases but there has been no report or patent related to the use of extract from cashew leaf or the active ingredients in the cashew leaf extract with the potential uses in the said fields.

This invention is aimed to improve health benefit of the cashew leaf extract by combined with some additional compounds that be able to control releasing of active compounds as the cashew leaf extract with encapsulated in the capsule.

### SUMMARY OF THE INVENTION

A composition containing cashew leaf extract comprises at least cashew leaf extract, magnesium stearate and maltodextrin wherein the said cashew leaf extract is extracted by water or alcohol or the combination of alcohol and water or other organic solvents or combination thereof, preferably the hydroalcoholic extracts. The composition containing cashew leaf extract can be used as the tonic and/or adaptogenic agents, for enhancing the efficiency of male sexual functions, for enhancing male sexual fertility, for improving cognitive functions.

The composition containing cashew leaf extract according to this invention is aimed to increase the commercialization value of cashew leaf which is rich of the health benefit from several nutrient contained in the cashew leaf such as increasing the function of neurological system related to monoamines especially dopamine which increase male sexual functions and also improving the cognitive function.

### BRIEF DESCRIPTION OF THE DRAWING

FIG. 1 The effect of hydro-alcoholic extracts of A. occidentale leaves extract on serum testosterone level of stress-exposed rats at 7 days and 14 days of treatment. Data were expressed as mean±S.E.M. (n=6/group). *, **, ***P-value <0.05, 0.01 and 0.001; compared with vehicle plus stress. ^{###}P-value <0.001; compared with control group.

FIG. 2 The effect of hydro-alcoholic extracts of A. occidentale leaves extract on serum corticosterone level of stress-exposed rats at 7 days and 14 days of treatment. Data were expressed as mean±S.E.M. (n=6/group). *, **P-value <0.05 and 0.01; compared with vehicle plus stress. ^{##, ###}P-value <0.01 and 0.001; compared with control group

FIG. 3 The effect of hydro-alcoholic extracts of A. occidentale leaves extract on tyrosine hydroxylase immunoreactive neurons in vental tegmental area of stress-exposed rats. Data were expressed as mean±S.E.M. (n=6/group). *, ***P-value <0.05 and 0.001; compared with vehicle plus stress. ^{#}P-value <0.05; compared with control group.

FIG. 4 The effect of hydro-alcoholic extracts of A. occidentale leaves extract on tyrosine hydroxylase immunoreactive neurons in core and shell of nucleus accumbens of stress-exposed rats. Data were expressed as mean±S.E.M. (n=6/group). *, **, ***P-value <0.05, 0.01 and 0.001; compared with vehicle plus stress. ^{#, ##}P-value <0.05 and 0.01;

FIG. 5 The effect of hydro-alcoholic extracts of A. occidentale leaves extract on phosphodiesterase-5 activity in penis of stress-exposed rats. Data were expressed as mean±S.E.M. (n=6/group). *, **, ***P-value <0.05, 0.01 and 0.01; compared with vehicle plus stress. ^{#}P-value <0.05; compared with control group

FIG. 6 The effect of hydro-alcoholic extracts of A. occidentale leaves extract on monoamine oxidase-B in medial preoptic area and nucleus accumbens of stress-exposed rats. Data were expressed as mean S.E.M. (n=6/group). *, ***P-value <0.05 and 0.001; compared with vehicle plus stress. ^{#, ###}P-value <0.05 and 0.001; compared with control group

FIG. 7 The effect of hydro-alcoholic extracts of A. occidentale leaves extract on endothelial nitric oxide synthase in penis of stress-exposed rats. Data were expressed as mean±S.E.M. (n=5/group). *P-value <0.05; compared with vehicle plus stress. ^{#}P-value <0.05; compared with control group

FIG. 8 The effect of hydro-alcoholic extracts of A. occidentale leaves extract on mounting latency of stress-exposed rats at baseline, after a single dose, 7 days and 14 days of treatment. Data were expressed as mean±S.E.M. (n=6/group). *, **, ***P-value <0.05, 0.01 and 0.001; compared with vehicle plus stress. ^{#, ##, ###}P-value <0.05, 0.01 and 0.001; compared with control group

FIG. 9 The effect of hydro-alcoholic extracts of A. occidentale leaves extract on mounting number of stress-exposed rats at baseline, after a single dose, 7 days and 14 days of treatment. Data were expressed as mean±S.E.M. (n=6/group). *P-value <0.05; compared with vehicle plus stress

FIG. 10 The effect of hydro-alcoholic extracts of A. occidentale leaves extract on intromission latency of stress-exposed rats at baseline, after a single dose, 7 days and 14 days of treatment. Data were expressed as mean±S.E.M. (n=6/group). *, **, ***P-value <0.05, 0.01 and 0.001; compared with vehicle plus stress. ^{#, ##, ###}P-value <0.05, 0.01 and 0.001; compared with control group.

FIG. 11 The effect of hydro-alcoholic extracts of A. occidentale leaves extract on intromission number of stress-exposed rats at baseline, after a single dose, 7 days and 14 days of treatment. Data were expressed as mean±S.E.M. (n=6/group). *, **P-value <0.05 and 0.01; compared with vehicle plus stress. ^{#, ##}P-value <0.05 and 0.01; compared with control group

FIG. 12 The effect of hydro-alcoholic extracts of A. occidentale leaves extract on ejaculation latency of stress-exposed rats at baseline, after a single dose, 7 days and 14 days of treatment. Data were expressed as mean±S.E.M. (n=6/group). *P-value <0.05; compared with vehicle plus stress. ^{#, ##}P-value <0.05 and 0.01; compared with control group

FIG. 13 The effect of hydro-alcoholic extract of A. occidentale leaves extract on ejaculation frequency of stress-exposed rats at baseline, after a single dose, 7 days and 14 days of treatment. Data were expressed as mean±S.E.M. (n=6/group). *, **P-value <0.05 and 0.01; compared with vehicle plus stress.

FIG. 14 Effect of ethanolic extract of A. occidentale leaves extract on human sperm. Values are mean±S.D.

FIG. 15 The effect of hydro-alcoholic extracts of A. occidentale leaves extract on histomorphology of rat testis stained with Haematoxylin and eosin (H&E). The cross section photographs showed the 1) seminiferous tubules (40× magnification) and 2) interstitial cells of Leydig (100× magnification), Sertoli cells (SC), spermatogonia (SG), primary spermatocytes (PS), spermatids (SP), sperm and Leydig cells (LC) in the following treatment groups: A) naïve control B) vehicle+stress C) Sildenafil citrate 5 mg/kg+stress D) Tianeptine 15 mg/kg+stress E)-G) A. occidentale at doses of 25, 100 and 200 mg/kg+stress, respectively.

### DETAILED DESCRIPTION OF THE INVENTION

A composition containing cashew leaf extract comprises at least cashew leaf extract, magnesium stearate and maltodextrin which each component has the following content;

| | |
|---|---|
| Cashew leaf extract | 50-90% w/w |
| magnesium stearate | 5-20% w/w |
| maltodextrin | 5-30% w/w |

The composition containing cashew leaf extract according to this invention further comprises pharmaceutical acceptable carriers.

The composition containing cashew leaf extract according to this invention wherein the cashew leaf extract is extracted by water or alcohol or the combination of alcohol and water (hydroalcoholic extract) or other organic solvents or combination thereof, preferably is hydroalcoholic extract. The said extract is obtained from the process using organic solvent.

A production method of the composition containing cashew leaf extract comprises the following steps;
a. Weighing each component in the composition recipe as the following cashew leaf extract, magnesium stearate and maltodextrin, then mixing all components in clean mixer in the rate of 100 round per min at least 30 min until the mixture powder become homogenous, then the mixture powder is dried at 70 degree Celsius for 2-5 hours and cool down at room temperature before add into the capsules by semi-automatic capsule filling machine or keep in the vacuum aluminium bag at −20 degree Celsius
b. Weighing the prepared mixture powder depend on the number of capsules, then separating capsules into body part and cap part, then arranging each part into specific tray which is the component of capsule filling machine, then pouring and spreading the mixture powder into the body part of capsule until the said mixture powder fully in the body part arranged on the tray, then packed with the compressor which is a component of the capsule filling machine until the mixture powder is fully filled in the body part, then assembling the packed body part of capsules containing cashew leaf extract with the cap part of the capsule arranged on the tray, then bringing the capsule sifted on the sieve to remove the dust, then keeping the capsule in the dry opaque plastic packages before quality testing according to united States Pharmacopoeia (USP) under U.S. Pharmacopeia National Formulary 2017 (USP 40 NF 35) volume 1

The invention discloses the composition containing the hydroalcoholic extract of cashew leaf (Anacardium occidentale). In this invention the constituent of the cashew leaf extract were identified as quercetin, myricetin, catechin, epicatechin, amentoflavone, tetramer of proanthocyanidin, quercetin glycosides, rutin, tannin, alkaloid and saponin. It was found that the stability of the extract is high even when the extract was stored at the temperature higher than a room temperature. More importantly, the biological activities of the extract changed slightly at the high temperature condition.

The toxicity of the cashew leaf extract is considered to be very low or safe for consumption with the LD50 of more than 2 gram/kg body weight. The toxicity study was performed in animal models using both male and female rat. The toxicity test results are shown in tables 1-14.

TABLE 1
Body weight of rat fed with cashew leaf extract at the
dosage of 2 gram/kg body weight.
Body weight (g)

| Group | n | Day 0 | Day 14 | % increase |
|---|---|---|---|---|
| Male rats | | | | |
| Control | 15 | 242.07 ± 3.87 | 259.79 ± 3.97 | 6.1 |
| Cashew leaf extract | 15 | 266.50 ± 7.02 | 282.71 ± 6.53 | 6.23 |
| (2 g/kg body weight) | | | | |
| Female rats | | | | |
| Control | 15 | 213.07 ± 2.84 | 228.43 ± 4.74 | 5.14 |
| Cashew leaf extract | 15 | 227.53 ± 3.39 | 239.79 ± 3.78 | 6.79 |
| (2 g/kg body weight) | | | | |

TABLE 2
Weight of internal organs of male rat fed with cashew leaf
extract at the dosage of 2 gram/kg body weight.

| | | Organ weight (g/kg BW, mean ± S.E.M.) | |
|---|---|---|---|
| | | | cashew leaf |
| | | | extract at the |
| | | | dosage of 2 g/kg |
| Visceral organ | | Control group | body weight |
| Lung | | 5.22 ± 0.28 | 5.14 ± 0.29 |
| Heart | | 3.44 ± 0.06 | 3.30 ± 0.05 |
| Liver | | 29.80 ± 0.82 | 30.12 ± 0.74 |
| Spleen | | 2.74 ± 0.10 | 2.72 ± 0.10 |
| Brain | | 5.71 ± 0.08 | 5.12 ± 0.11 |
| Pancreas | | 4.69 ± 0.37 | 5.22 ± 0.48 |
| Stomach | | 6.47 ± 0.20 | 7.36 ± 0.26 |
| Thymus | | 2.37 ± 0.14 | 1.86 ± 0.09 |
| Kidney | left | 3.79 ± 0.15 | 3.54 ± 0.11 |
| | right | 3.89 ± 0.09 | 3.49 ± 0.07 |
| Testis | left | 8.90 ± 0.25 | 8.21 ± 0.47 |
| | right | 8.99 ± 0.20 | 8.05 ± 0.42 |
| Salivary gland | left | 0.45 ± 0.02 | 0.45 ± 0.02 |
| | right | 0.48 ± 0.02 | 0.44 ± 0.02 |
| Adrenal grand | left | 0.21 ± 0.01 | 0.18 ± 0.01 |
| | right | 0.22 ± 0.01 | 0.16 ± 0.02 |
| Number of rats | | 15 | 15 |

TABLE 3
Weight of internal organs of female rat fed with cashew leaf
extract at the dosage of 2 gram/kg body weight.

| | | Organ weight (g/kg BW, mean ± S.E.M.) | |
|---|---|---|---|
| | | | cashew leaf |
| | | | extract at the |
| | | | dosage of 2 g/kg |
| Visceral organ | | Control group | body weight |
| Lung | | 6.80 ± 0.54 | 6.02 ± 0.39 |
| Heart | | 3.48 ± 0.10 | 3.42 ± 0.10 |
| Liver | | 30.54 ± 1.73 | 28.15 ± 1.07 |
| Spleen | | 2.77 ± 0.11 | 2.96 ± 0.19 |
| Brain | | 5.97 ± 0.10 | 6.29 ± 0.10 |
| Pancreas | | 4.76 ± 0.34 | 5.19 ± 0.39 |
| Stomach | | 7.04 ± 0.18 | 7.32 ± 0.16 |
| Thymus | | 2.13 ± 0.12 | 1.95 ± 0.09 |
| Kidney | left | 3.44 ± 0.29 | 3.41 ± 0.08 |
| | right | 3.52 ± 0.29 | 3.38 ± 0.25 |
| Ovary | left | 0.66 ± 0.05 | 0.73 ± 0.06 |
| | right | 0.70 ± 0.07 | 0.78 ± 0.07 |
| Salivary gland | left | 0.50 ± 0.03 | 0.45 ± 0.03 |
| | right | 0.47 ± 0.04 | 0.46 ± 0.02 |
| Adrenal grand | left | 0.27 ± 0.29 | 0.23 ± 0.01 |
| | right | 0.24 ± 0.04 | 0.23 ± 0.01 |
| Number of rats | | 15 | 15 |

TABLE 4
Histological change of male rat fed with the cashew leaf
extract at the dosage of 2 gram/kg body weight

| | Group (Mean ± S.E.M.) | | |
|---|---|---|---|
| | | | cashew leaf |
| | | | extract at the |
| | Reference | Control | dosage of 2 g/kg |
| CBC | value | group | body weight |
| WBC | 0.96-7.88 | 2.25 ± 0.29 | 3.48 ± 4.09* |
| (× 10³ cells/mm³) | | | |
| RBC | 7.16-9.24 | 8.14 ± 0.31 | 8.06 ± 0.55 |
| (× 10⁶ cells/mm³) | | | |
| Hemoglobin (g/dl) | 13.7-17.2 | 12.69 ± 0.47 | 12.90 ± 0.07 |
| Hematocrit (%) | 38.5-49.2 | 39.19 ± 1.52 | 42.66 ± 0.78 |
| MCV (fL) | 50.3-57 | 48.21 ± 0.72 | 53.45 ± 0.67*** |
| MCH (pg/red cell) | 17.6-20.3 | 15.62 ± 0.22 | 15.99 ± 0.90 |
| MCHC (g/dl RBC) | 33.2-37.8 | 32.45 ± 0.39 | 29.96 ± 0.15 |
| Platelet | 599-1144 | 666.85 ± 145.81 | 1229.47 ± 114.54 |
| (× 10³ cells/mm³) | | | |
| Neutrophil (%) | 8.8-43.8 | 14.31 ± 1.60 | 16.10 ± 2.63 |
| Lymphocyte (%) | 48.9-88.1 | 78.53 ± 1.23 | 77.53 ± 2.20 |
| Monocyte (%) | 1-3.6 | 3.983 ± 1.21 | 3.14 ± 0.72 |
| Eosinophil (%) | 0.3-4.7 | 0.23 ± 0.06 | 1.17 ± 0.10 |
| Basophil (%) | 0-0.7 | 2.99 ± 1.24 | 2.06 ± 0.78 |
| Number of rats | 15 | 15 | |

*, ***p-value <.05 and .01 respectively, compared to treatment

TABLE 5
Histological change of male rat fed with the cashew leaf
extract at the dosage of 2 gram/kg body weight

| | Group (Mean ± S.E.M.) | | |
|---|---|---|---|
| | | | cashew leaf |
| | | | extract at the |
| | Reference | Control | dosage of 2 g/kg |
| CBC | value | group | body weight |
| WBC | 0.96-7.88 | 3.78 ± 0.27 | 3.07 ± 0.27 |
| (× 10³ cells/mm³) | | | |
| RBC | 7.16-9.24 | 8.30 ± 0.16 | 8.50 ± 0.18 |
| (× 10⁶ cells/mm³) | | | |
| Hemoglobin (g/dl) | 13.7-17.2 | 15.11 ± 0.29 | 15.42 ± 0.30 |
| Hematocrit (%) | 38.5-49.2 | 44.23 ± 0.85 | 43.96 ± 0.78 |
| MCV (fL) | 50.3-57 | 53.11 ± 0.60 | 51.79 ± 0.40 |
| MCH (pg) | 17.6-20.3 | 18.21 ± 0.12 | 18.15 ± 0.11 |
| MCHC (g/dl) | 33.2-37.8 | 34.17 ± 0.23 | 35.06 ± 0.17** |
| Platelet | 599-1144 | 688.33 ± 48.75 | 787.07 ± 46.85 |
| (× 10³ cells/mm³) | | | |
| Neutrophil (%) | 8.8-43.8 | 15.16 ± 1.99 | 21.93 ± 1.19 |
| Lymphocyte (%) | 48.9-88.1 | 78.72 ± 1.99 | 73.92 ± 1.59 |
| Monocyte (%) | 1-3.6 | 3.35 ± 0.82 | 1.89 ± 0.53 |
| Eosinophil (%) | 0.3-4.7 | 1.42 ± 0.34 | 1.573 ± 0.64 |
| Basophil (%) | 0-0.7 | 1.35 ± 0.73 | 0.54 ± 0.37 |
| Number of rats | 15 | 15 | |

TABLE 6
Clinical chemistry change of male rat fed with the cashew
leaf extract at the dosage of 2 gram/kg body weight

| | | | cashew leaf |
|---|---|---|---|
| | | | extract at the |
| Blood | Reference | Control | dosage of 2 g/kg |
| chemistry | value | group | body weight |
| BUN (mg/dl) | 10.7-20 | 23.36 ± 1.09 | 23.87 ± 1.10 |
| Creatinine (mg/dl) | 0.3-0.5 | 0.56 ± 0.04 | 0.45 ± 0.02* |
| Cholesterol (mg/dl) | 37-95 | 60.36 ± 2.04 | 60.27 ± 2.56 |
| Triglyceride (mg/dl) | 27-160 | 38.29 ± 2.92 | 42.13 ± 4.73 |
| ALT (U/L) | 19-48 | 45.50 ± 3.47 | 31.27 ± 3.27** |
| AST (U/L) | 63-175 | 98.00 ± 6.40 | 102.60 ± 4.27 |
| ALP (U/L) | 36-131 | 78.14 ± 6.50 | 48.33 ± 7.28** |
| Total Bilirubin (mg/dl) | 0.04-0.2 | 0.18 ± 0.01 | 0.18 ± 0.03 |

*, **p-value <.05 and .01 respectively, compared to treatment

TABLE 7
Clinical chemistry change of female rat fed with the cashew
leaf extract at the dosage of 2 gram/kg body weight

| | Group (Mean ± S.E.M.) | | |
|---|---|---|---|
| | | | cashew leaf |
| | | | extract at the |
| Blood | Reference | Control | dosage of 2 g/kg |
| chemistry | value | group | body weight |
| UN (mg/dl) | 11.7-25 | 32.86 ± 1.07 | 19.50 ± 0.73*** |
| Creatinine (mg/dl) | 0.3-0.6 | 0.54 ± 0.03 | 0.36 ± 0.02*** |
| Cholesterol (mg/dl) | 23-97 | 60.50 ± 1.63 | 65.93 ± 3.15 |
| Triglyceride (mg/dl) | 16-175 | 57.36 ± 6.14 | 34.43 ± 1.49 |
| ALT (U/L) | 14-64 | 30.00 ± 1.43 | 34.21 ± 1.76** |
| AST (U/L) | 64-222 | 84.29 ± 3.55 | 109.71 ± 3.47*** |
| ALP (U/L) | 18-62 | 51.71 ± 8.19 | 62.07 ± 3.14 |
| Total Bilirubin (mg/dl) | 0.07-0.21 | 0.25 ± 0.03 | 0.20 ± 0.03 |

*, **p-value <.05 and .01 respectively, compared to treatment

Sub-chronic toxicity study of the cashew leaf extract according to this invention showed that both male and female rat have no significant growth as shown in table 8. Moreover, no significant change of the weight of internal organs of male rat as shown in table 9 but in female rat which were fed with 100 mg of extract/kg of body weight the weight of brain and size of stomach were reduced significantly as shown in table 10.

TABLE 8
Body weight of rat fed with cashew leaf extract at the dosage
of 20, 100, 500 mg/kg body weight for 90 days (n = 15/group)

| | Body weight (g) | | |
|---|---|---|---|
| | | | Weight |
| | | | increase at |
| Group | Day 1 | Day 100 | Day 100 |
| Control male | | | |
| | 208.20 ± 3.83 | 245.87 ± 7.41 | 20.41 |
| Cashew leaf extract | 235.00 ± 3.76 | 283.13 ± 8.77 | 17.65 |
| 20 mg/kg | | | |
| Cashew leaf extract | 238.33 ± 3.76 | 281.07 ± 4.53 | 18.13 |
| 100 mg/kg | | | |
| Cashew leaf extract | 231.67 ± 4.65 | 274.93 ± 4.74 | 19.25 |
| 500 mg/kg | | | |
| Control female | | | |
| | 335.33 ± 6.98 | 420.73 ± 7.78 | 26.540 |
| Cashew leaf extract | 359 ± 3.98 | 451.67 ± 9.44 | 25.770 |
| 20 mg/kg | | | |
| Cashew leaf extract | 356.60 ± 4.66 | 451.53 ± 7.67 | 26.680 |
| 100 mg/kg | | | |
| Cashew leaf extract | 355.54 ± 5.98 | 448.46 ± 11.60 | 26.580 |
| 500 mg/kg | | | |

TABLE 9
Internal organ weight of male rat fed with cashew leaf extract at the
dosage of 20, 100, 500 mg/kg body weight for 90 days (n = 15/group)

| | | Internal organ weight (g/kg body weight) | | | |
|---|---|---|---|---|---|
| | | | AO 20 | AO 100 | AO 500 |
| | | Control | mg/kgBW | mg/kgBW | mg/kgBW |
| Lung | | 8.20 ± 0.96 | 6.88 ± 0.72 | 7.59 ± 0.71 | 6.06 ± 0.25 |
| Heart | | 3.20 ± 0.12 | 3.08 ± 0.10 | 0.10 ± 0.08 | 3.37 ± 0.13 |
| Liver | | 24.64 ± 1.20 | 24.51 ± 0.86 | 0.86 ± 2.85 | 26.37 ± 2.56 |
| Spleen | | 2.26 ± 0.17 | 2.27 ± 0.23 | 0.23 ± 0.06 | 2.50 ± 0.35 |
| Brain | | 3.45 ± 0.07 | 3.28 ± 0.09 | 0.09 ± 0.07 | 3.53 ± 0.10 |
| Pancreases | | 4.67 ± 0.36 | 4.11 ± 0.27 | 0.27 ± 0.29 | 4.21 ± 0.30 |
| Stomach | | 5.03 ± 0.25 | 5.14 ± 0.31 | 0.31 ± 0.16 | 5.32 ± 0.27 |
| Thymus | | 0.96 ± 0.08 | 1.04 ± 0.05 | 0.05 ± 0.09 | 1.06 ± 0.10 |
| Kidney | Left | 3.03 ± 0.15 | 3.13 ± 0.14 | 0.14 ± 0.07 | 3.14 ± 0.25 |
| | Right | 4.59 ± 1.51 | 2.98 ± 0.11 | 0.11 ± 0.08 | 2.97 ± 0.16 |
| Testis | Left | 4.76 ± 0.20 | 4.55 ± 0.13 | 0.13 ± 0.11 | 4.48 ± 0.40 |
| | Right | 4.79 ± 0.21 | 4.67 ± 0.15 | 0.15 ± 0.10 | 4.59 ± 0.21 |
| Saliva | Left | 0.17 ± 0.01 | 0.16 ± 0.01 | 0.01 ± 0.01 | 0.16 ± 0.02 |
| gland | Right | 0.15 ± 0.01 | 0.16 ± 0.01 | 0.01 ± 0.01 | 0.17 ± 0.01 |
| Pituitary | Left | 0.35 ± 0.02 | 0.34 ± 0.02 | 0.02 ± 0.02 | 0.37 ± 0.02 |
| gland | Right | 0.33 ± 0.02 | 0.35 ± 0.03 | 0.03 ± 0.01 | 0.36 ± 0.02 |

TABLE 10
Internal organ weight of female rat fed with cashew leaf extract at the
dosage of 20, 100, 500 mg/kg body weight for 90 days (n = 15/group)

| | | Internal organ weight (g/kg body weight) | | | |
|---|---|---|---|---|---|
| | | | AO 20 | AO 100 | AO 500 |
| | | Control | mg/kgBW | mg/kgBW | mg/kgBW |
| Lung | | 11.21 ± 0.81 | 10.42 ± 0.96 | 8.85 ± 0.82 | 10.73 ± 1.65 |
| Heart | | 4.58 ± 0.44 | 3.92 ± 0.09 | 3.74 ± 0.11 | 3.83 ± 0.19 |
| Liver | | 34.72 ± 1.16 | 32.74 ± 0.92 | 31.97 ± 1.21 | 32.41 ± 1.19 |
| Spleen | | 3.19 ± 0.12 | 4.81 ± 1.88 | 2.96 ± 0.18 | 3.03 ± 0.33 |
| Brain | | 5.51 ± 0.01 | 5.34 ± 0.09 | 5.23* ± 0.15 | 5.23 ± 0.09 |
| Pancreases | | 6.29 ± 0.36 | 7.28 ± 0.44 | 6.43 ± 0.68 | 5.69 ± 0.47 |
| Stomach | | 7.29 ± 0.32 | 6.82 ± 0.49 | 6.42 ± 0.18 | 6.77 ± 0.24 |
| Thymus | | 1.37 ± 0.08 | 1.18 ± 0.09 | 1.17 ± 0.09 | 1.47 ± 0.09 |
| Kidney | Left | 3.69 ± 0.10 | 3.62 ± 0.13 | 3.32 ± 0.25 | 3.46 ± 0.11 |
| | Right | 3.74 ± 0.11 | 3.57 ± 0.10 | 3.45 ± 0.15 | 3.48 ± 0.09 |
| Testis | Left | 0.68 ± 0.06 | 0.68 ± 0.05 | 0.72 ± 0.18 | 0.57 ± 0.04 |
| | Right | 0.68 ± 0.06 | 0.55 ± 0.06 | 0.72 ± 0.17 | 0.56 ± 0.02 |
| Saliva | Left | 0.39 ± 0.04 | 0.49 ± 0.04 | 0.60 ± 0.15 | 0.56 ± 0.16 |
| gland | Right | 0.41 ± 0.04 | 0.44 ± 0.04 | 0.44 ± 0.03 | 0.39 ± 0.03 |
| Pituitary | Left | 0.33 ± 0.04 | 0.30 ± 0.03 | 0.24 ± 0.02 | 0.25 ± 0.01 |
| gland | Right | 0.30 ± 0.03 | 0.26 ± 0.02 | 0.24 ± 0.02 | 0.24 ± 0.02 |

TABLE 11
Histological change of male rat fed with cashew leaf extract at the
dosage of 20, 100, 500 mg/kg body weight for 90 days (n = 15/group)

| | Group (Mean ± S.E.M.) | | | | |
|---|---|---|---|---|---|
| | Reference | Control | AO 20 | AO 100 | AO 500 |
| CBC | values | group | mg/kgBW | mg/kgBW | mg/kgBW |
| WBC | 1.98-11.06 | 4.93 ± 0.28 | 4.29 ± 0.27 | 3.03 ± 0.15*** | 3.23 ± 0.18*** |
| (× 10³ ells/mm³) | | | | | |
| RBC | 7.62-9.99 | 8.10 ± 0.67 | 8.27 ± 0.51 | 8.54 ± 0.14 | 8.29 ± 0.13 |
| (× 10⁶ cells/mm³) | | | | | |
| Hemoglobin (g/dl) | 13.60-17.40 | 13.90 ± 1.08 | 13.83 ± 0.97 | 14.87 ± 0.24 | 14.52 ± 0.17 |
| Hematocrit (%) | 38.50-52.00 | 44.31 ± 2.71 | 42.49 ± 2.48 | 44.34 ± 0.83 | 43.45 ± 0.31 |
| MCV (fL) | 46.30-56.20 | 57.32 ± 2.80 | 52.09 ± 1.46 | 51.95 ± 0.56 | 52.49 ± 0.66 |
| MCH (pg) | 16.30-19.50 | 17.45 ± 0.38 | 16.64 ± 0.40 | 17.44 ± 0.15 | 17.51 ± 0.18 |
| MCHC (g/dl) | 31.90-38.50 | 30.90 ± 0.90 | 32.05 ± 0.73 | 33.55 ± 0.13* | 33.42 ± 0.29 |
| Platelet | 547-1253 | 876.31 ± 53.90 | 943.53 ± 68.91 | 750.93 ± 29.57* | 746.62 ± 49.05 |
| (× 10³ cells/mm³) | | | | | |
| Neutrophil (%) | 9.00-49.30 | 25.30 ± 1.07 | 25.55 ± 1.37 | 27.79 ± 1.03 | 27.67 ± 1.59 |
| Lymphocyte (%) | 44.70-87.10 | 69.45 ± 1.20 | 68.06 ± 1.96 | 67.06 ± 1.04 | 66.60 ± 1.78 |
| Monocyte (%) | 1.00-3.60 | 4.22 ± 0.47 | 4.09 ± 0.72 | 2.36 ± 0.46 | 3.45 ± 0.93 |
| Eosinophil (%) | 0.40-4.00 | 1.04 ± 0.35 | 1.37 ± 0.47 | 2.80 ± 0.66 | 2.25 ± 0.52 |
| Basophil (%) | 0-0.60 | 0.00 ± 0.00 | 0.01 ± 0.01 | 0.00 ± 0.00 | 0.03 ± 0.03 |

TABLE 12
Histological change of female rat fed with cashew leaf extract at the
dosage of 20, 100, 500 mg/kg body weight for 90 days (n = 15/group)

| | Group (Mean ± S.E.M.) | | | | |
|---|---|---|---|---|---|
| | Reference | Control | AO 20 | AO 100 | AO 500 |
| CBC | values | group | mg/kgBW | mg/kgBW | mg/kgBW |
| WBC | 1.98-11.06 | 5.02 ± 0.41 | 4.07 ± 0.33* | 4.03 ± 0.37* | 2.91 ± 0.25*** |
| (× 10³ ells/mm³) | | | | | |
| RBC | 7.62-9.99 | 7.77 ± 0.70 | 8.23 ± 0.20 | 8.17 ± 0.14 | 9.17 ± 0.23 |
| (× 10⁶ cells/mm³) | | | | | |
| Hemoglobin (g/dl) | 13.60-17.40 | 13.35 ± 1.12 | 14.92 ± 0.31 | 14.68 ± 0.29 | 16.11 ± 0.33* |
| Hematocrit (%) | 38.50-52.00 | 42.70 ± 2.73 | 45.78 ± 0.95 | 45.50 ± 1.06 | 51.04 ± 0.98* |
| MCV (fL) | 46.30-56.20 | 57.96 ± 3.02 | 55.77 ± 0.95 | 55.67 ± 0.84 | 55.89 ± 0.88 |
| MCH (pg) | 16.30-19.50 | 17.50 ± 0.41 | 18.15 ± 0.15 | 17.97 ± 0.23 | 17.61 ± 0.20 |
| MCHC (g/dl) | 31.90-38.50 | 30.73 ± 0.98 | 32.62 ± 0.30 | 32.32 ± 0.18 | 31.59 ± 0.36 |
| Platelet | 547-1253 | 857.64 ± 60.92 | 701.47 ± 34.29* | 691.31 ± 53.72* | 583.86 ± 57.11* |
| (× 10³ cells/mm³) | | | | | |
| Neutrophil (%) | 9.00-49.30 | 25.91 ± 2.37 | 23.89 ± 1.88 | 21.35 ± 1.73 | 27.76 ± 2.86 |
| Lymphocyte (%) | 44.70-87.10 | 69.96 ± 2.16 | 71.63 ± 1.75 | 73.59 ± 1.68 | 65.51 ± 2.65 |
| Monocyte (%) | 1.00-3.60 | 3.23 ± 0.56 | 3.95 ± 0.58 | 4.08 ± 0.66 | 5.07 ± 1.01 |
| Eosinophil (%) | 0.40-4.00 | 0.90 ± 0.18 | 1.53 ± 0.32 | 0.98 ± 0.09 | 1.64 ± 0.28 |
| Basophil (%) | 0-0.60 | 0.00 ± 0.00 | 0.08 ± 0.08 | 0.00 ± 0.00 | 0.01 ± 0.01 |

As shown in tables 11 and 12, the cashew leaf extract may cause some changes in histological index, but they are in the normal ranges.

TABLE 13
Clinical chemistry data of male rat fed with cashew leaf extract at the
dosage of 20, 100, 500 mg/kg body weight for 90 days (n = 15/group)

| | Group (Mean ± S.E.M.) | | | | |
|---|---|---|---|---|---|
| Blood | Reference | Control | AO 20 | AO 100 | AO 500 |
| Chemistry | values | group | mg/kgBW | mg/kgBW | mg/kgBW |
| BUN (mg/dl) | 10.7-20 | 19.91 ± 0.70 | 18.68 ± 0.79 | 17.95 ± 0.78 | 19.37 ± 1.02 |
| Creatinine(ml/dl) | 0.3-0.5 | 0.50 ± 0.02 | 0.50 ± 0.03 | 0.47 ± 0.02 | 0.45 ± 0.02 |
| Cholesterol(ml/dl) | 37-95 | 64.60 ± 3.29 | 66.00 ± 2.81 | 57.00 ± 2.46 | 60.33 ± 4.72 |
| Triglyceride(ml/dl) | 27-160 | 75.92 ± 8.24 | 48.86 ± 7.79* | 46.40 ± 3.29*** | 46.20 ± 4.27** |
| ALT (U/L) | 19-48 | 123.67 ± 7.89 | 142.93 ± 7.43 | 144.64 ± 2.53 | 140.14 ± 13.11 |
| AST (U/L) | 63-175 | 49.53 ± 7.89 | 55.93 ± 5.63 | 38.40 ± 3.23 | 45.07 ± 3.07 |
| ALP (U/L) | 36-131 | 66.33 ± 5.14 | 82.60 ± 7.64 | 54.07 ± 4.49 | 62.50 ± 5.27 |
| Total | 0.04-0.2 | 0.1 ± 0.001 | 0.12 ± 0.0145 | 0.1 ± 4E-18 | 0.1 ± 4E-18 |
| Bilirubin (mg/dl) | | | | | |

TABLE 14
Clinical chemistry data of male rat fed with cashew leaf extract at the
dosage of 20, 100, 500 mg/kg body weight for 90 days (n = 15/group)

| | Group (Mean ± S.E.M.) | | | | |
|---|---|---|---|---|---|
| Blood | Reference | Control | AO 20 | AO 100 | AO 500 |
| Chemistry | values | group | mg/kgBW | mg/kgBW | mg/kgBW |
| BUN (mg/dl) | 11.7-25 | 22.68 ± 0.59 | 22.69 ± 0.65 | 21.66 ± 0.59 | 22.77 ± 0.46 |
| Creatinine (ml/dl) | 0.3-0.6 | 0.48 ± 0.02 | 0.49 ± 0.02 | 0.49 ± 0.02 | 0.54 ± 0.03 |
| Cholesterol (ml/dl) | 23-97 | 54.20 ± 4.67 | 52.47 ± 2.51 | 55.73 ± 2.98 | 58.73 ± 2.77 |
| Triglyceride (ml/dl) | 16-175 | 62.00 ± 6.51 | 52.13 ± 4.54 | 74.00 ± 4.84 | 64.67 ± 5.36 |
| ALT (U/L) | 14-64 | 141.80 ± 6.16 | 139.93 ± 5.10 | 142.64 ± 7.16 | 142.29 ± 6.79 |
| AST (U/L) | 64-222 | 35.67 ± 2.71 | 43.80 ± 3.88 | 39.00 ± 2.36 | 44.80 ± 5.76 |
| ALP (U/L) | 18-62 | 51.67 ± 7.28 | 60.33 ± 6.92 | 62.20 ± 3.43 | 93.33 ± 32.99 |
| Total | 0.07-0.21 | 0.11 ± 0.007 | 0.12 ± 0.014 | 0.1 ± 0.001 | 0.1 ± 0.001 |
| Bilirubin (mg/dl) | | | | | |

### Biological Activities of the Cashew Leaf Extract:

The cashew leaf extract showed the strong activity for reduction of triglyceride level in male rat when fed with the said extract at the concentration of 20, 100 and 200 mg/kg body weight compared to the control rat but not in female rat as shown in tables 13 and 14. Moreover, the consumption of rat during the study of this invention was not changed significantly when compared to control rat.

### Pharmacological Activities of the Cashew Leaf Extract

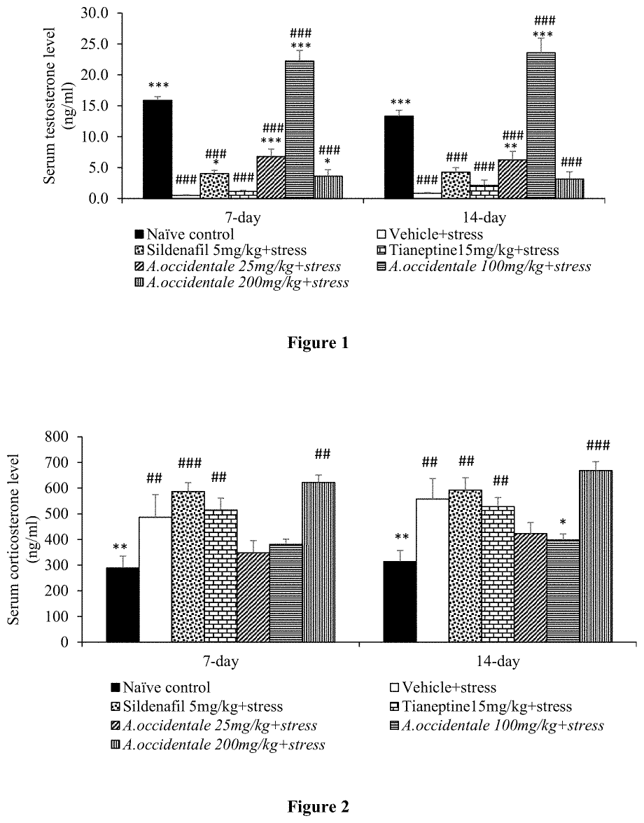

The cashew leaf extract according to this invention showed the pharmacological activities in increasing the function of neurological system related to monoamines especially dopamine which increase sexual function of male rat population. The said activities are caused by the inhibition of enzymes that inhibit the function of monoamines especially dopamine, increasing of testosterone, reduction of corticosterone as shown in FIG. 2.

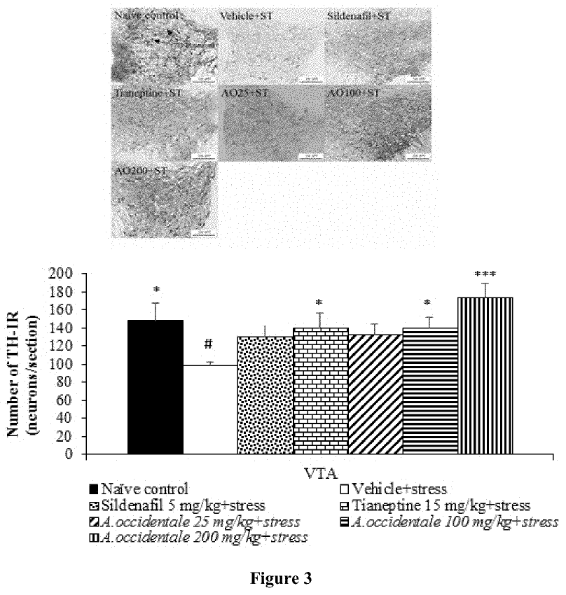

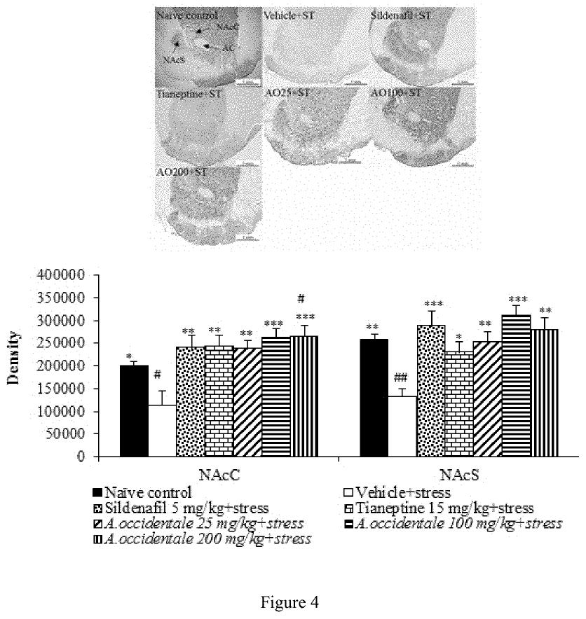

Moreover, the cashew leaf extract according to this invention can also increase the density of dopamine neuron cells in the region of ventral tegmental area (VTA) and nucleus accumbent (NAc) of the brain as shown in FIGS. 3 and 4. The ventral tegmental area (VTA) and nucleus accumbent (NAc) of the brain are responsible for libido.

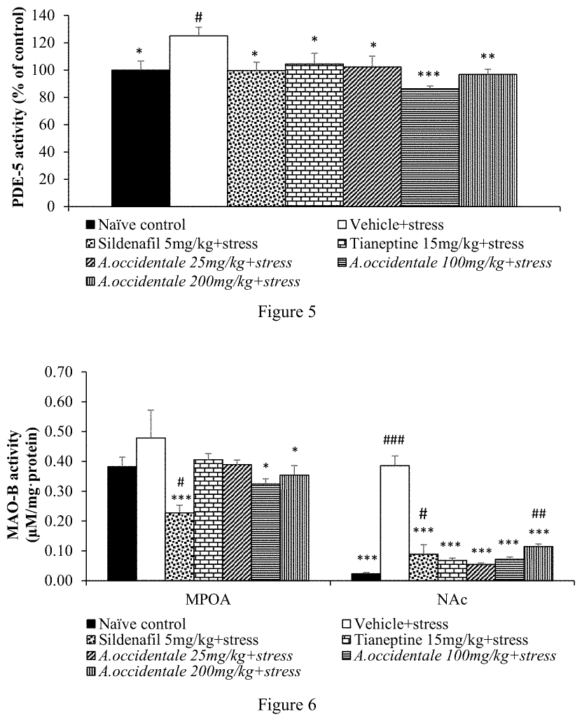

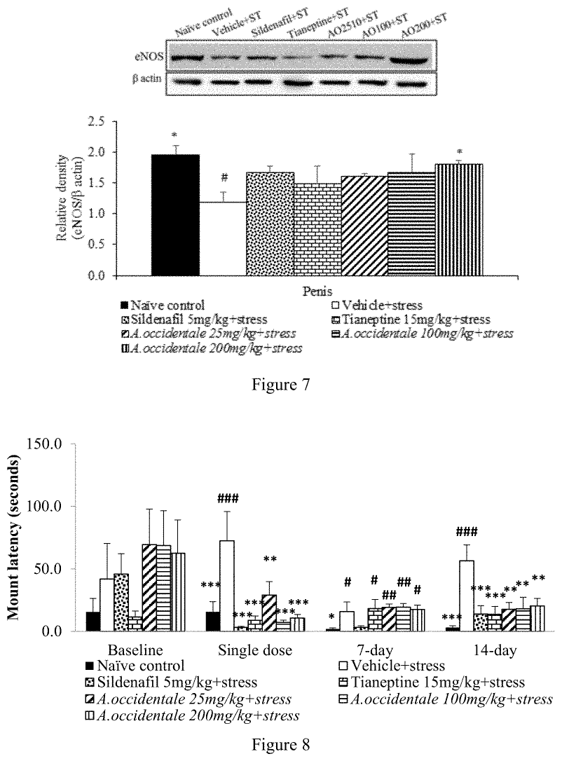

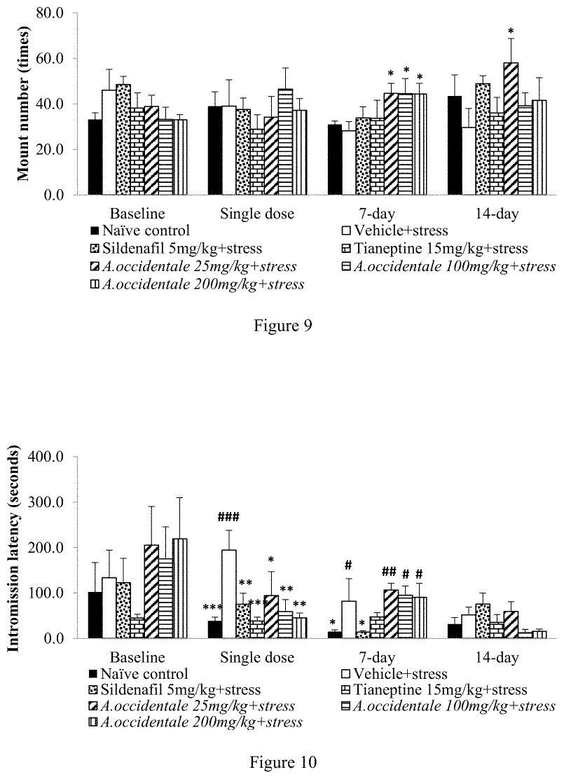

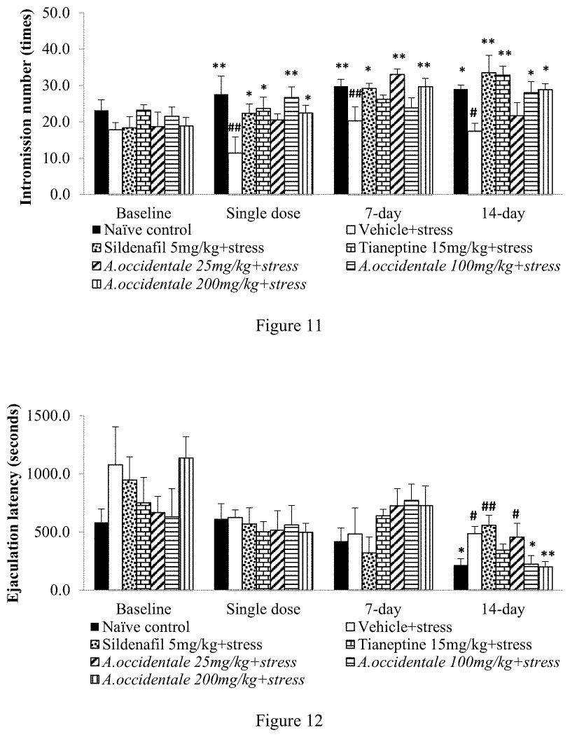

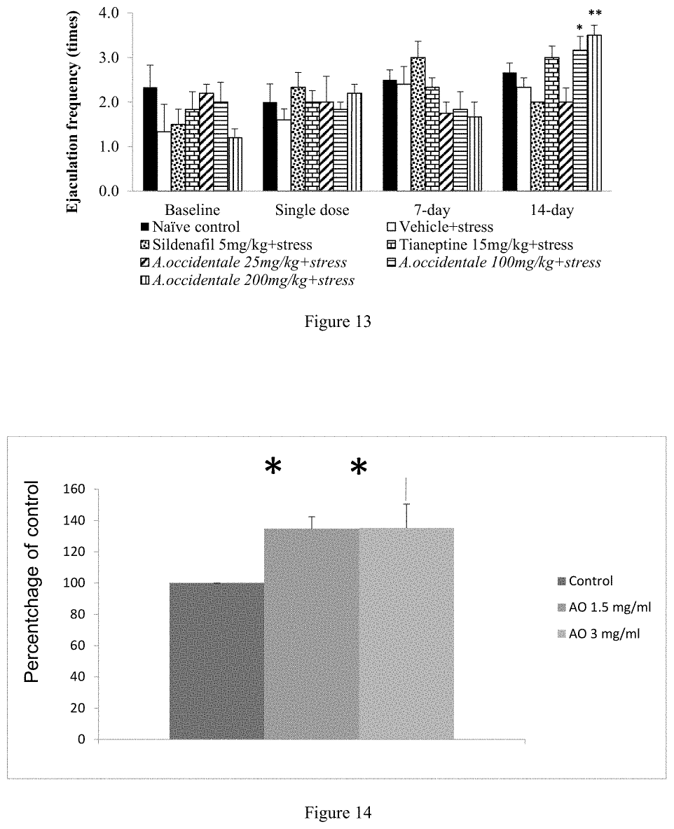

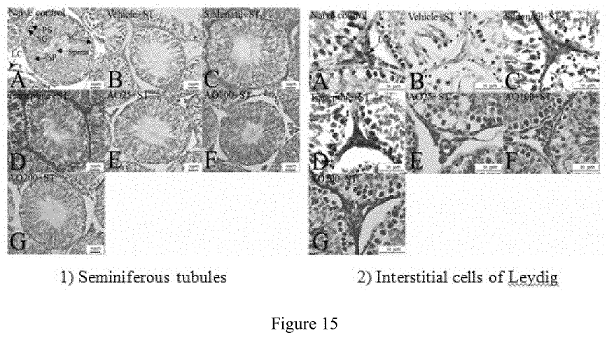

Furthermore, the cashew leaf extract according to this invention can also increase the blood circulation which cause penis erection by inhibiting phosphodiesterase type 5 enzyme as shown in FIG. 5. The said extract can also inhibit the activity of enzymes involving with inhibition of the monoamines especially dopamine in medial preoptic area (mPOA) and nucleus accumben (NAc) as shown in FIG. 6 and increase the amount of endothelial nitric oxide synthase (eNOS) in penis as shown in FIG. 7. It is therefore increasing the libido as shown in FIGS. 8-13.

The cashew leaf extract according to this invention was obtained from the hydroalcoholic extraction process with 95% ethanol or the other extraction process e.g. supercritical fluid extraction, microwave extraction or ultra-sonic extraction or other method alike.
1. The said extract comprises of phenolic or flavonoid compounds comprise of gallic acid or rutin or quercetin or combination thereof. The phenolic compounds content of the said extract is ranging from 75 to 300 ug GAF/mg extract and the flavonoid compounds content is ranging from 1 to 75 ug quercetin/mg extract. It is important to note that the gallic acid and rutin and quercitin ratio is ranging from 20-140:5-20:0.05-10. In order to obtain the pharmacological activities of the composition containing the said extract, the concentration of the said extract is ranging from 100-500 mg/unit of serving.

The composition according to this invention can be used as tonic and/or adaptogenic agent. The functions of tonic and adaptogenic is caused by exerting the effect at multi-target organs including but not limited to the reducing of stress hormone or glucocorticoids or sympathetic enhancing effects.

The composition according to this invention can also be used for enhancing the efficiency of male sexual functions. the efficiency of male sexual functions is caused by exerting the effect at multi-target organs including but not limited to increasing of testosterone and/or increasing of dopaminergic activities in brain and/or suppressing phosphodiesterase type 5 and/or reducing of oxidative stress status and/or enhancing nitric oxide formation in male sexual organ.

The composition according to this invention can also be used for enhancing male sexual fertility. The enhancing male sexual fertility is caused by increasing of testosterone level and/or decreasing oxidative stress status in male sexual organs and/or increasing of spermatogenesis.

The composition according to this invention can also be used for improving cognitive functions. The improving cognitive functions by enhancing cholinergic functions and/or decreasing oxidative stress status and/or neurogenesis and/or enhancing signal transduction via mitogen activated protein kinase (MAPK) in a brain.

The scope of this invention also includes food, beverage or food supplement or health products comprise of the said composition. Food, beverage or food supplement or health products according to this invention may include but not limited to food, beverage or food supplement or health products in the form of tablet, capsule, effervescent granule or tablet, granule, powder, gel, jelly, gum, candy, spray, film, patch, cream, ointment, lotion or similar.

Although, this invention has been disclosed in the context of certain embodiments and examples, it will be understood by those skilled in the art that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the disclosed invention. Thus, it is intended that the scope of the present invention herein disclosed should not be limited by the particular disclosed embodiments described above, but should be determined only by a fair reading of the claims that follow.

1. A composition containing cashew leaf extract comprises at least cashew leaf extract, magnesium stearate and maltodextrin.

2. The composition containing cashew leaf extract according to claim 1 wherein comprises as the following content;

| | |
|---|---|
| Cashew leaf extract | 50-90% w/w |
| Magnesium stearate | 5-20% w/w |
| Maltodextrin | 5-30% w/w |

3. The composition containing cashew leaf extract according to claim 1, wherein said composition can be further comprised pharmaceutical acceptable carriers.

4. The composition containing cashew leaf extract according to claim 1, wherein cashew leaf extract is extracted by water or alcohol or the combination of alcohol and water or other organic solvents or combination thereof.

5. The composition containing cashew leaf extract according to claim 1, wherein said extract is hydroalcoholic extract.

6. The composition containing cashew leaf extract according to claim 1, wherein the said extract is obtained from the process using organic solvent.

7. The composition containing cashew leaf extract according to claim 1, wherein the said composition can be used as tonic and/or adaptonic agent.

8. The composition containing cashew leaf extract according to claim 1, wherein the said composition can be used for enhancing the efficiency of male sexual functions.

9. The composition containing cashew leaf extract according to claim 1, wherein the said composition can be used for enhancing male sexual fertility.

10. The composition containing cashew leaf extract according to claim 1, wherein the said composition can be used for improving cognitive functions.

11. A production method of composition containing cashew leaf extract comprises the following steps;
a. Weighing each component in the composition recipe as the following cashew leaf extract, magnesium stearate and maltodextrin, then mixing all components in clean mixer in the rate of 100 round per min at least 30 min until the mixture powder become homogenous, then the mixture powder is dried at 70 degree Celsius for 2-5 hours and cool down at room temperature before add into the capsules by semi-automatic capsule filling machine or keep in the vacuum aluminium bag at −20 degree Celsius,
b. Weighing the prepared mixture powder depend on the number of capsules, then separating capsules into body part and cap part, then arranging each part into specific tray which is the component of capsule filling machine, then pouring and spreading the mixture powder into the body part of capsule until the said mixture powder fully in the body part arranged on the tray, then packed with the compressor which is a component of the capsule filling machine until the mixture powder is fully filled in the body part, then assembling the packed body part of capsules containing cashew leaf extract with the cap part of the capsule arranged on the tray, then bringing the capsule sifted on the sieve to remove the dust, then keeping the capsule in the dry opaque plastic packages before quality testing according to united States Pharmacopoeia (USP) under U.S. Pharmacopeia National Formulary 2017 (USP 40 NF 35) volume 1.

12. The food, beverage or food supplement or health products comprises of the said composition according to claim 1.

13. The food, beverage or food supplement or health products according claim 12 wherein the food, beverage or food supplement or health products can be in the form of tablet, capsule, effervescent granule or tablet, granule, powder, gel, jelly, gum, candy, spray, film, patch, cream, ointment, lotion or similar.
